(19) United States
(12) Patent Application Publication
(10) Pub. No.: US 2004/0186577 A1
(43) Pub. Date: Sep. 23, 2004

(54) In situ artificaial disc replacements and other prosthetic components

(76) Inventor: Bret A. Ferree, Cincinnati, OH (US)

Correspondence Address: John G. Posa, Gifford, Krass, Groh, Sprinkle,, Anderson & Citkowski, P.C., 280 N. Old Woodward Ave., Suite 400, Birmingham, MI, 48009-5394, US

(21) Appl. No.: 10/767,194

(22) Filed: Jan. 29, 2004

Related U.S. Application Data
(60) Provisional application No. 60/443,324, filed on Jan. 29, 2003.

(51) Int. Cl. A61F 2/44

(52) U.S. Cl. 623/17.16; 623/17.11

(57) ABSTRACT

Multi-component artificial disc replacements (ADRs) facilitate in situ assembly within a disc space. In contrast to single-component ADRs, which use endplates constructed of a single material, assembled ADRs according to the invention allow the use of more than one material, even for the endplates themselves in certain embodiments. As such, materials with good wear characteristics such as chrome cobalt can be combined with materials such as Nitinol exhibiting other desirable characteristics such as the elasticity or shape-memory properties. Devices according to the invention can be used for other joints of the body, such as prosthetic knees and hips.

### REFERENCE TO RELATED APPLICATION

This application claims priority from U.S. Provisional Patent Application Serial No. 60/443,324, filed Jan. 29, 2003, the entire content of which is incorporated herein by reference.

### FIELD OF THE INVENTION

This invention relates generally to prosthetic components and, in particular, to artificial disc replacements (ADRs) and other implants.

### BACKGROUND OF THE INVENTION

Premature or accelerated disc degeneration is known as degenerative disc disease. A large portion of patients suffering from chronic low back pain are thought to have this condition. As the disc degenerates, the nucleus and annulus functions are compromised. The nucleus becomes thinner and less able to handle compression loads. The annulus fibers become redundant as the nucleus shrinks. The redundant annular fibers are less effective in controlling vertebral motion. The disc pathology can result in: 1) bulging of the annulus into the spinal cord or nerves; 2) narrowing of the space between the vertebra where the nerves exit; 3) tears of the annulus as abnormal loads are transmitted to the annulus and the annulus is subjected to excessive motion between vertebra; and 4) disc herniation or extrusion of the nucleus through complete annular tears.

Current surgical treatments of disc degeneration are destructive. One group of procedures removes the nucleus or a portion of the nucleus; lumbar discectomy falls in this category. A second group of procedures destroy nuclear material; Chymopapin (an enzyme) injection, laser discectomy, and thermal therapy (heat treatment to denature proteins) fall in this category. A third group, spinal fusion procedures either remove the disc or the disc's function by connecting two or more vertebra together with bone. These destructive procedures lead to acceleration of disc degeneration. The first two groups of procedures compromise the treated disc. Fusion procedures transmit additional stress to the adjacent discs. The additional stress results in premature disc degeneration of the adjacent discs.

Prosthetic disc replacement offers many advantages. The prosthetic disc attempts to eliminate a patient's pain while preserving the disc's function. Current prosthetic disc implants, however, either replace the nucleus or the nucleus and the annulus. Both types of current procedures remove the degenerated disc component to allow room for the prosthetic component. Although the use of resilient materials has been proposed, the need remains for further improvements in the way in which prosthetic components are incorporated into the disc space, and in materials to ensure strength and longevity. Such improvements are necessary, since the prosthesis may be subjected to 100,000,000 compression cycles over the life of the implant.

Existing artificial disc replacement (ADR) devices use simple, one piece, endplate (EP) components. Although robust, one piece ADR EPs have certain deficiencies. For example, wide spacers are often placed between the ADR EPs. U.S. Pat. No. 5,401,269 describes the use of a convex spacer that is forced into concavities within the ADR EPs. Excessive distraction may be required to insert the wide spacers. Furthermore, the spacers can extrude from the ADR EPs if too narrow of spacer is used. FIG. 1 is a sagittal cross section of a prior-art ADR similar to that taught in U.S. Pat. No. 5,401,269. Single ADR EPs articulate with a biconvex spacer.

U.S. Pat. No. 5,258,031 eliminates the risk of extruding the spacer component by eliminating the spacer component from the design. The ADR EPs of '031 patent articulate. A ball projection from one EP fits into a socket in the other ADR component. The device of the '031 patent is inserted fully assembled. Fully assembled ADRs such as that described in the '031 patent can not be rely on press fit projections from the ADR EPs to prevent extrusion of the device. FIG. 2 is a sagittal cross section of a prior art ADR similar to that taught in U.S. Pat. No. 5,238,031. The articulating components of the ADR are placed directly on the vertebral endplates.

U.S. Pat. No. 5,562,738 teaches methods and devices which permit insertion into “cups” that can be press fit into the vertebrae. The '738 patent describes the use of “body-compatible adhesive, tape, solder attachment, or clip mechanism” between the articulating components and the cups. The '738 patent does not teach how the “clip mechanism” works. Furthermore, it would be difficult, if not impossible, to insert the articulating components after press fitting the “cup” components by the methods and devices shown in some of the figures in the '738 patent. For example, the articulating components of FIGS. 7 and 14 are too tall to fit through the opening between the press fit “cups”. In addition, the screw connecting the “cups” of FIGS. 20 and 21 could not be inserted into the articulating components after press fitting the “cups” into the vertebrae. The opening for the screw would be covered by the vertebral endplates. Furthermore, the perpendicular orientation of the screw require insert of the screw through cup into the articulating component, outside of the disc space.

### SUMMARY OF THE INVENTION

This invention broadly resides in multi-component artificial disc replacements (ADRs) that facilitate assembly within a disc space. In the preferred embodiments, the ADR apparatus includes an endplate that articulates with a cooperating component, and wherein the endplate, or the endplate and the cooperating component, are physically configured for assembly within an intervertebral disc space.

In contrast to single-component ADRs, which use endplates constructed of a single material, assembled ADRs according to the invention allow the use of more than one material, even for the endplates themselves in certain embodiments. As such, materials with good wear characteristics such as chrome cobalt can be combined with materials such as Nitinol exhibiting other desirable characteristics such as the elasticity or shape-memory properties. Devices according to the invention can be used for other joints of the body, such as prosthetic knees and hips.

A method of implanting a prosthesis such as an artificial disc replacement (ADR) into an intervertebral disc space includes the provision of an endplate constructed from first and second components. The first component is then installed into an intervertebral disc space, and the second component is installed by attaching the second component to the first component, thereby assembling the endplate in situ.

Again, the first and second components may be composed of dissimilar materials, and the apparatus may further include a spacer component which is also assembled in situ.

### BRIEF DESCRIPTION OF THE DRAWINGS

FIG. 1 is a sagittal cross-section of a prior-art artificial disc replacement (ADR);

FIG. 2 is a sagittal cross-section of another prior-art ADR;

FIG. 3A is a sagittal cross-section of one embodiment of ADR endplates according to the present invention;

FIG. 3B is an exploded sagittal cross-section of the ADR drawn in FIG. 3A;

FIG. 3C is a coronal cross-section of the ADR drawn in FIG. 3A;

FIG. 3D is a coronal cross-section of an alternative embodiment of the ADR drawn in FIG. 3C;

FIG. 4 is a sagittal cross-section of an alternative embodiment of the ADR;

FIG. 5A is a sagittal cross-section of the first step in the sequence of inserting the ADR drawn in FIG. 4;

FIG. 5B is a sagittal cross-section of the second step in the sequence of inserting the ADR drawn in FIG. 4;

FIG. 5C is a sagittal cross-section of the third step in the sequence of inserting the ADR drawn in FIG. 4;

FIG. 6A is a sagittal cross-section of another embodiment of an ADR according to the present invention;

FIG. 6B is a sagittal cross-section of the ADR drawn in FIG. 6A with the hinged ADR components press fit into the vertebrae;

FIG. 6C is a sagittal cross-section of the ADR drawn in FIG. 6A;

FIG. 7A is a sagittal cross section of an alternative embodiment of the ADR drawn in FIG. 6A;

FIG. 7B is a view of the front of the ADR drawn in FIG. 7A with the press fit components rotated into one another;

FIG. 7C is a view of the front of the ADR drawn in FIG. 7C with the press fit components in an extended position;

FIG. 8A is the view of the front of an assembled ADR EP with an alternative attachment mechanism;

FIG. 8B is the view of the front of an assembled ADR EP with an alternative attachment mechanism;

FIG. 9 is the view of the top of yet a further alternative removable ADR EP component according to the invention, wherein the spring projections are on the sides of the component;

FIG. 10 is a view of the front of an assembled ADR EP. The removable component can fit with slots in the press fit component;

FIG. 11A is a coronal cross section through an ADR EP;

FIG. 11B is a coronal cross section of an alternative embodiment of the ADR drawn in FIG. 11A;

FIG. 12A is a sagittal cross-section of an alternative embodiment of the ADR;

FIG. 12B is a sagittal cross-section of an alternative embodiment of the ADR drawn in FIG. 12A;

FIG. 12C is a sagittal cross-section of an alternative embodiment of the ADR drawn in FIG. 12A;

FIG. 13 is a sagittal cross-section through an alternative embodiment of the ADR EP;

FIG. 14 is a sagittal cross-section through an alternative embodiment of the ADR;

FIG. 15 is a coronal cross-section through a different alternative embodiment of the invention;

FIG. 16A is a sagittal cross-section of an alternative embodiment of the ADR;

FIG. 16B is an exploded sagittal cross-section of the ADR drawn in FIG. 16A;

FIG. 16C is an axial cross-section of the top ADR EP drawn in FIG. 16A;

FIG. 16D is a coronal cross-section of the ADR drawn in FIG. 16A;

FIG. 17 is an axial cross-section of the spacer of FIG. 16A and a tool used to hold the spacer;

FIG. 18A is an axial cross-section of an alternative embodiment of the device;

FIG. 18B is an axial cross-section of the ADR drawn in FIG. 18A;

FIG. 19A is a coronal cross-section of an alternative embodiment of the device;

FIG. 19B is a coronal cross-section of the ADR drawn in FIG. 19A;

FIG. 20A is a coronal cross-section through yet a different alternative embodiment of the invention;

FIG. 20B is a coronal cross section of an alternative embodiment of the ADR drawn in FIG. 20A;

FIG. 20C is a coronal cross section of an alternative embodiment of the ADR drawn in FIG. 20A;

FIG. 21A is a sagittal cross-section of the spine and another embodiment of the ADR;

FIG. 21B is a drawing of the next step in the sequence of insertion of the ADR drawn in FIG. 21A;

FIG. 21C is a sagittal cross-section of the spine and the completed ADR drawn in FIG. 21A;

FIG. 22A is a sagittal cross-section of the spine and another embodiment of the ADR;

FIG. 22B is a sagittal cross-section of the spine and the next step in the insertion of the ADR drawn in FIG. 22A;

FIG. 22C is a sagittal cross-section of the spine and the next step in the insertion of the ADR drawn in FIG. 22B;

FIG. 22D is a sagittal cross-section of the spine and the next step in the insertion of the ADR drawn in FIG. 22C; and

FIG. 22E is a sagittal cross-section of the spine and the completed ADR drawn in FIG. 22D.

### DETAILED DESCRIPTION OF THE INVENTION

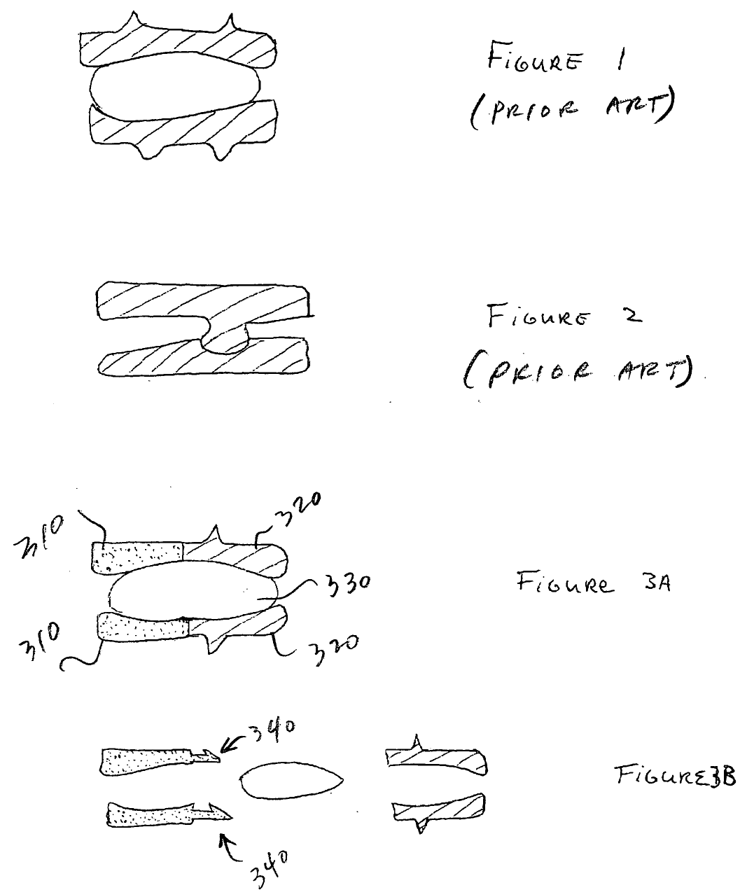

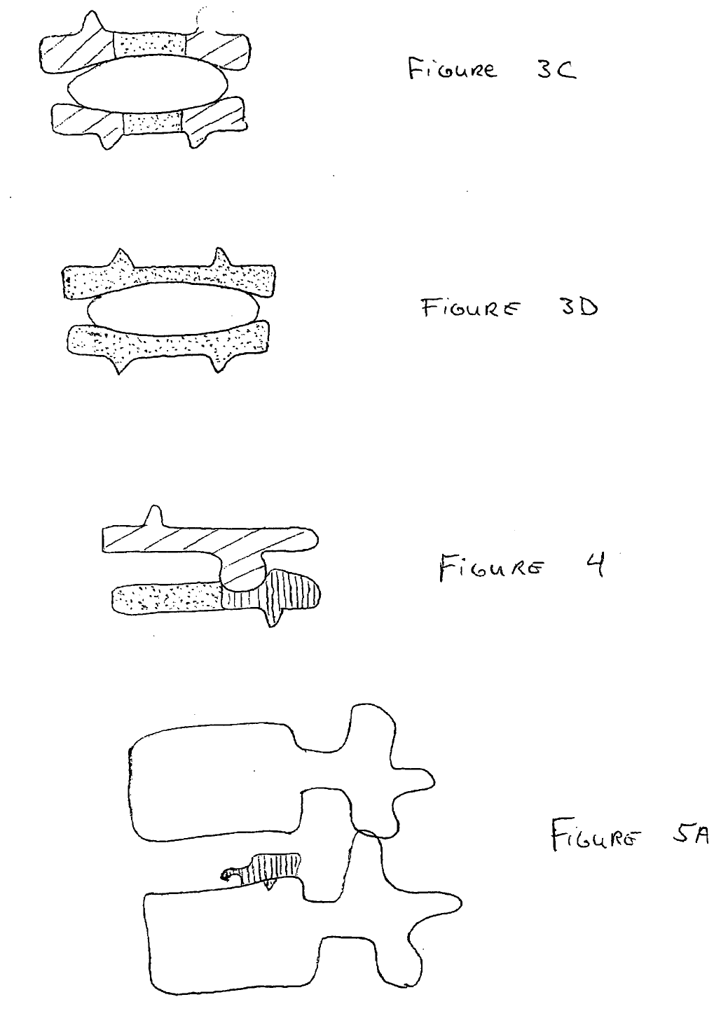

Having discussed the prior-art configurations of FIGS. 1 and 2, the reader's attention is directed to FIG. 3A, which is a sagittal cross section of one embodiment of ADR endplates according to this invention. Removable portions of the ADR EP (310) are assembled to the press-fit components of the ADR EPs (320), after insertion of a biconvex spacer 330. FIG. 3B is an exploded sagittal cross section of the ADR drawn in FIG. 3A. Spring projections 340 from the removable portion of the ADR EPs fit into corresponding holes (not visible) within the press fit components of the ADR EPs. FIG. 3C is a coronal cross section of the ADR drawn in FIG. 3A. The removable component of the ADR EP fits into a slot within the press fit component of the ADR EPs.

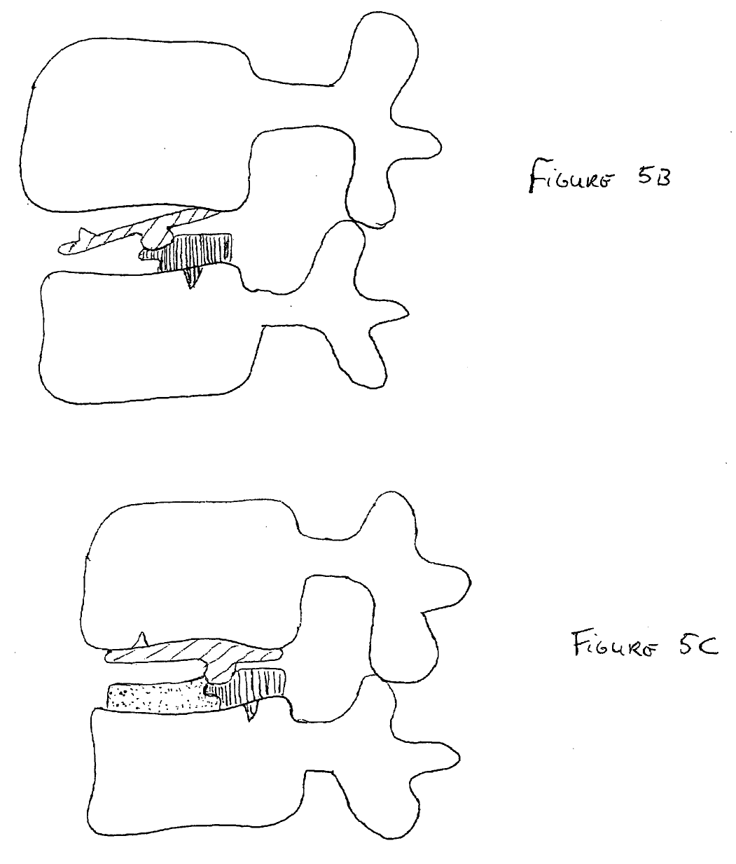

FIG. 3D is a coronal cross section of an alternative embodiment of the invention, wherein the removable ADR EP components occupy the entire side of the ADR through which the spacer is inserted. FIG. 4 is a sagittal cross section of an alternative embodiment of the ADR. Three components, two of which are press fit, are assembled within the disc space.

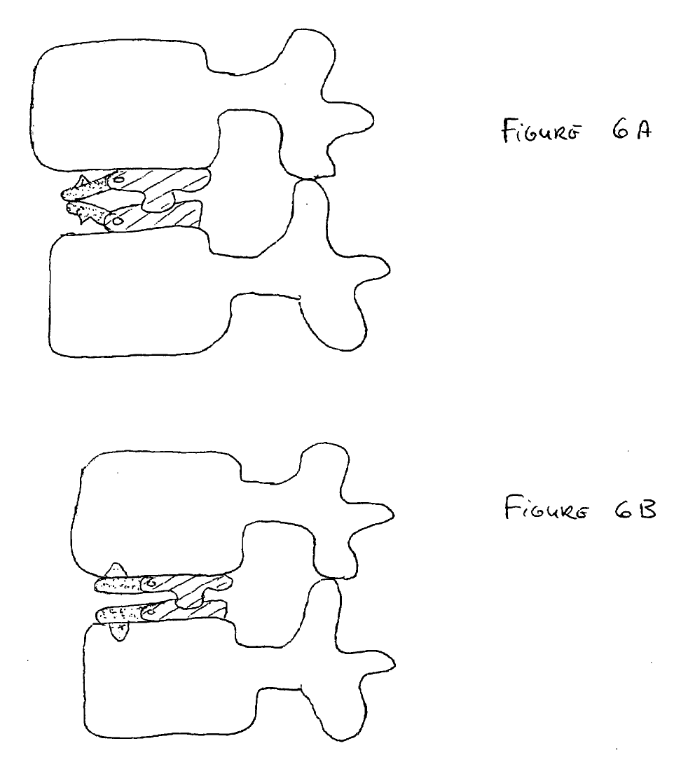

FIG. 5A is a sagittal cross section of the first step in the sequence of inserting the ADR drawn in FIG. 4. The first component is press fit into one of the vertebrae. FIG. 5B is a sagittal cross section of the second step in the sequence of inserting the ADR drawn in FIG. 4. The second ADR component is press fit into the second vertebra. FIG. 5C is a sagittal cross section of the third step in the sequence of inserting the ADR drawn in FIG. 4. The third component is attached to the first component. Spring projections such as those illustrated in FIG. 3B can be used to connect the two components.

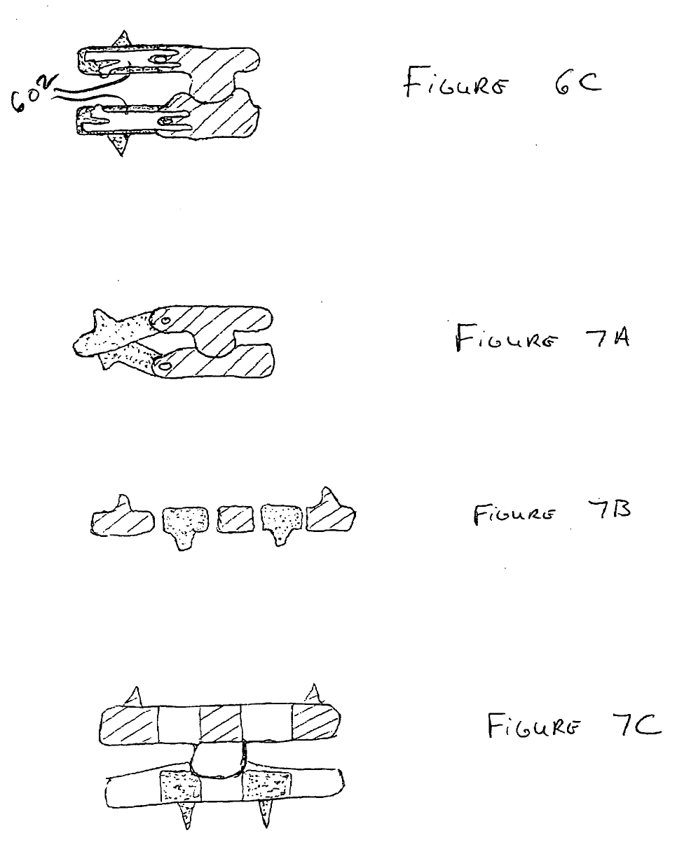

FIG. 6A is a sagittal cross section of another embodiment of an ADR according to the invention, wherein hinged components are press fit into the vertebrae after insertion of the ADR into the disc space. FIG. 6B is a sagittal cross section of the ADR drawn in FIG. 6A with the hinged ADR components press fit into the vertebrae. FIG. 6C is a sagittal cross section of the ADR drawn in FIG. 6A. Removable members 602 can be placed across the hinge joints to prevent rotation of the joints.

FIG. 7A is a sagittal cross section of an alternative embodiment of the ADR drawn in FIG. 6A. The press fit ADR components have interdigitating slots that allow the press fit components to collapse within one another. The press fit spikes can be longer in this embodiment of the device. FIG. 7B is a view of the front of the ADR drawn in FIG. 7A with the press fit components rotated into one another. FIG. 7C is a view of the front of the ADR drawn in FIG. 7C with the press fit components in an extended position.

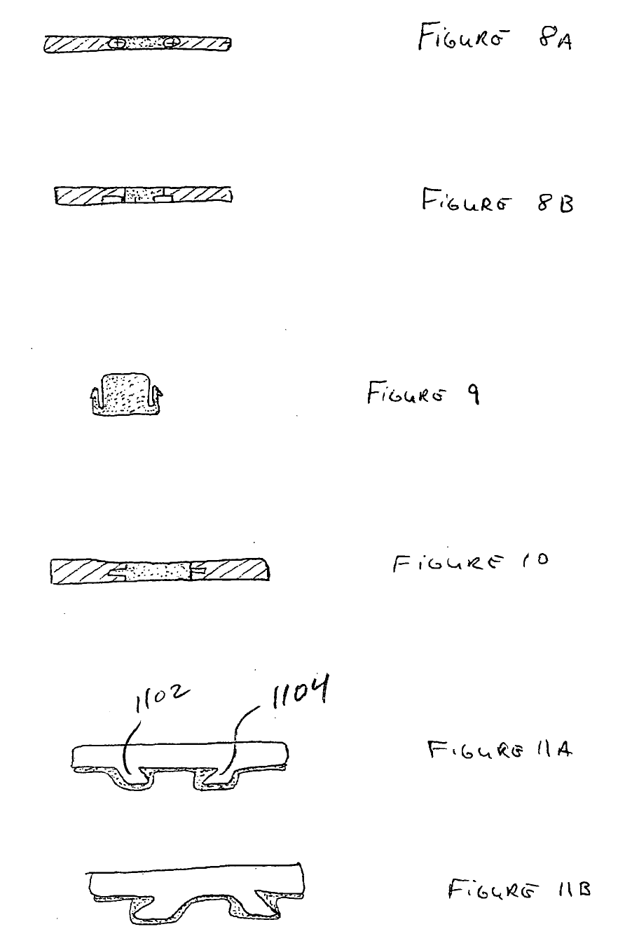

FIG. 8A is the view of the front of an assembled ADR EP with an alternative attachment mechanism. Screws are threaded into the ADR EP to lock the removable ADR component in position. The screws may have threads that deform slightly to prevent the screws from loosening. FIG. 8B is the view of the front of an assembled ADR EP with an alternative attachment mechanism. Rotating members are used to lock the removable ADR component in position. The rotating member on the left side of the drawing is in the open position. The rotating member on the right side of the drawing is in the closed position.

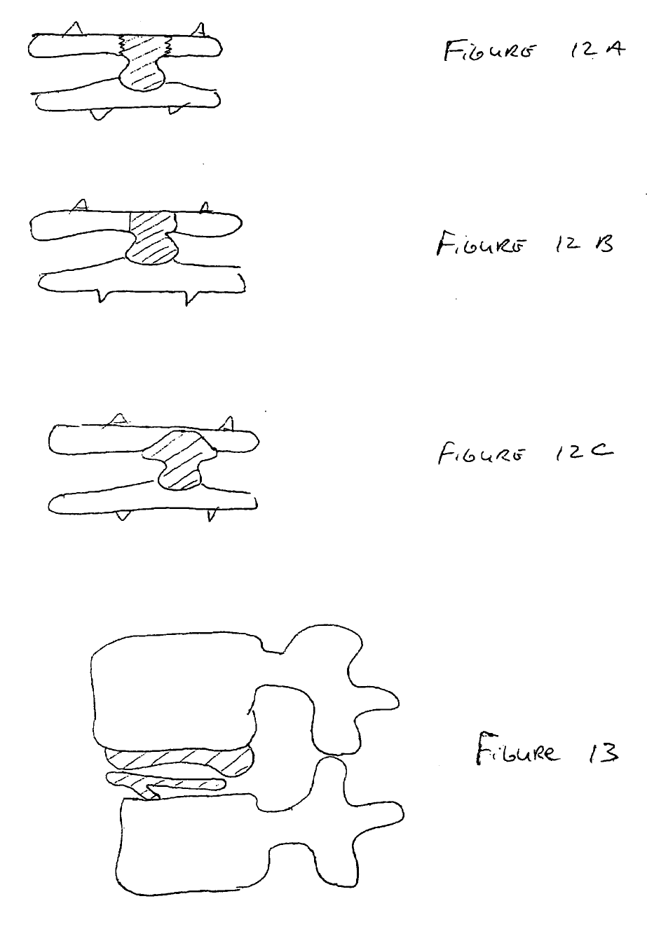

FIG. 9 is the view of the top of yet a further alternative removable ADR EP component according to the invention, wherein the spring projections are on the sides of the component. FIG. 10 is a view of the front of an assembled ADR EP. The removable component can fit with slots in the press fit component.

FIG. 11A is a coronal cross section through an ADR EP. Converging projections 1102, 1104 are cemented to the vertebral EPs. Converging projections improve the strength of the ADR EP—vertebra junction. FIG. 11B is a coronal cross section of an alternative embodiment including diverging projections cemented into the vertebrae to improve the strength of the ADR EP—vertebra junction.

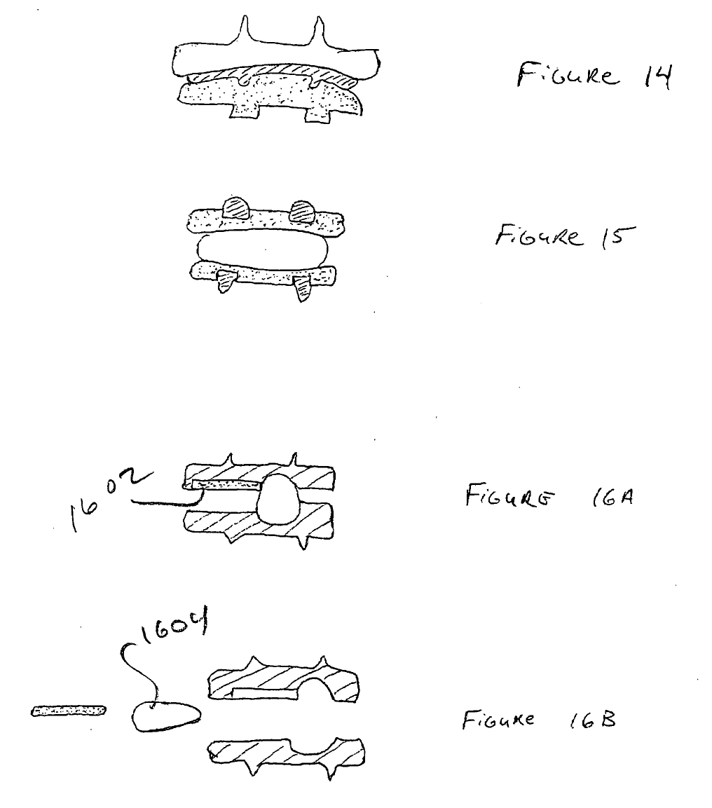

FIG. 12A is a sagittal cross section of an alternative embodiment of the ADR. An articulating component of one material is treaded into an ADR EP of a second material. FIG. 12B is a sagittal cross section of an alternative embodiment wherein the articulating component is press fit into the ADR EP. FIG. 12C is a sagittal cross section of an alternative embodiment wherein the articulating component is attached to the ADR EP through a Morse taper joint.

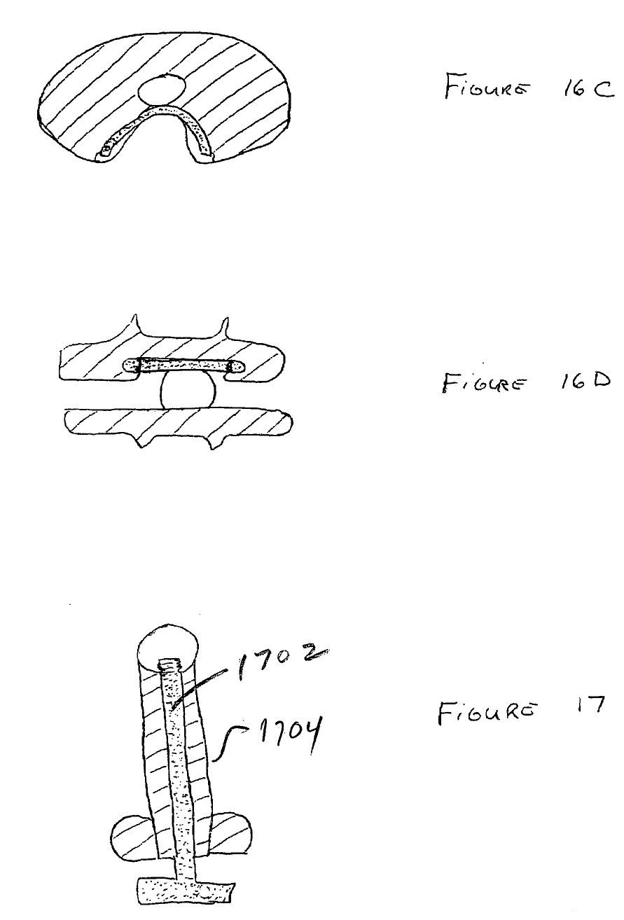

FIG. 13 is a sagittal cross section through an alternative embodiment of the ADR EP. The projection from the ADR EP is slanted in the direction of insertion, to allow the use of longer projections. The ADR EP is simultaneously slid under the second ADR EP and press fit into the vertebra.

FIG. 14 is a sagittal cross section through an alternative embodiment of an ADR. An ADR EP of one material is attached to an articulating component of a second material. For example, an articulating component of chrome cobalt could be attached to an ADR EP component of Nitinol or other shape-memory material. The Nitinol projections from the ADR EP could change shape to diverge or converge after insertion in the disc space. The elasticity of the Nitinol component would also allow the ADR EP to reversibly deform with spinal movement.

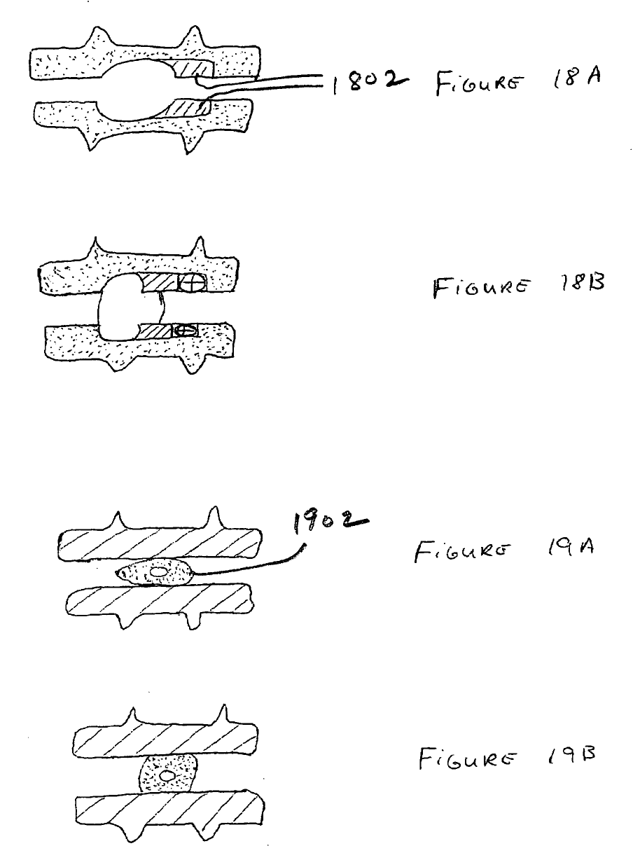

FIG. 15 is a coronal cross section through a different alternative embodiment of the invention, wherein projections of one material are placed into ADR EPs of a second material. FIG. 16A is a sagittal cross section of an alternative embodiment of an ADR wherein a removable clip component 1602 holds a removable spacer component 1604 in position between the ADR EPs. FIG. 16B is an exploded sagittal cross section of the ADR drawn in FIG. 16A. FIG. 16C is an axial cross section of the top ADR EP drawn in FIG. 16A. The removable clip fits into a slot in the ADR EP. FIG. 16D is a coronal cross section of the ADR drawn in FIG. 16A.

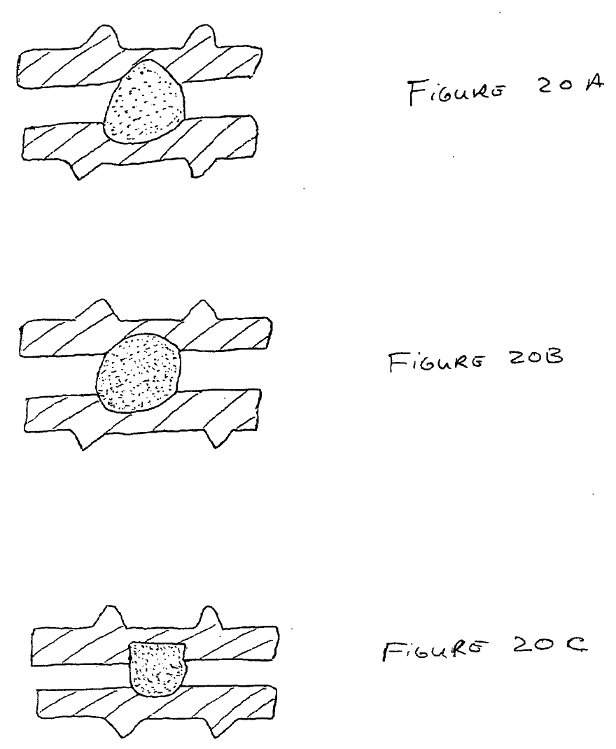

FIG. 17 is an axial cross section of the spacer of FIG. 16A and a tool used to hold the spacer. A component 1702 of the tool is threaded into the spacer component. A second component 1704 of the tool is fitted over the spacer to prevent rotation of the spacer while inserting and removing the threaded component of the tool.

FIG. 18A is an axial cross section of an alternative embodiment of the device. Sliding components 1802 are shown in a position that facilitates insertion of the spacer component. FIG. 18B is an axial cross section of the ADR drawn in FIG. 18A, with the sliding components in a position that blocks extrusion of the spacer component. The sliding components can be held in the closed position with screws that are threaded into the ADR EPs. The screw threads can deform to prevent screw loosening.

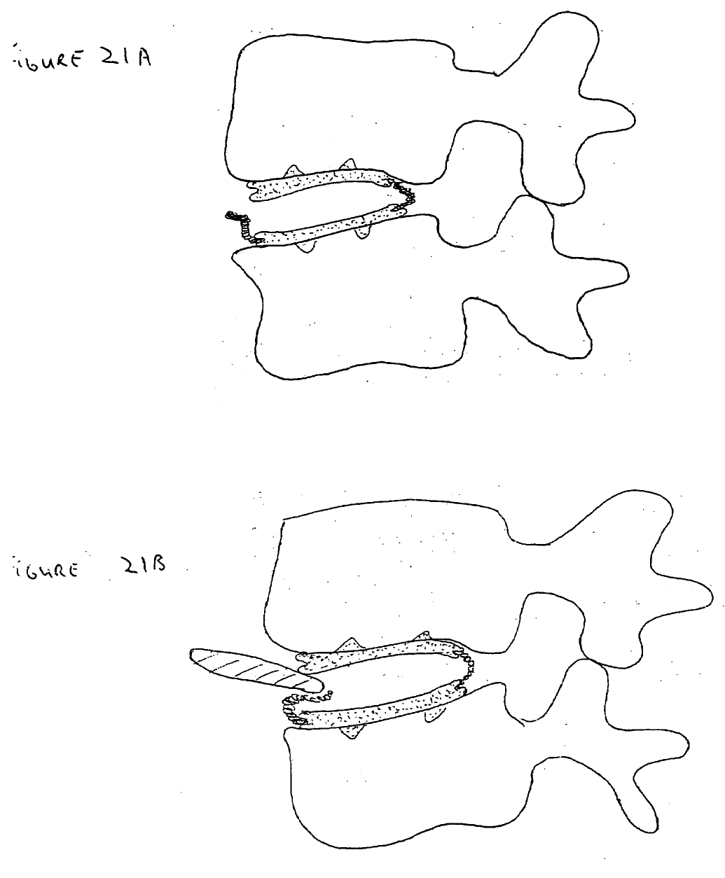

FIG. 19A is a coronal cross section of an alternative embodiment with a spacer component 1902 shown during insertion between the ADR EPs. The spacer component is inserted with its long axis parallel to the opening in the ADR EPs. The hole in the center of the spacer component can be used by an insertion tool. The hole within the spacer component may also allow the spacer component to reversibly deform with spinal movement. FIG. 19B is a coronal cross section of the ADR drawn in FIG. 19A. The spacer component is shown in its final position. Rotation of the spacer component 90 degrees from the insertion position to the final position cams the ADR EPs apart to distract the vertebrae.

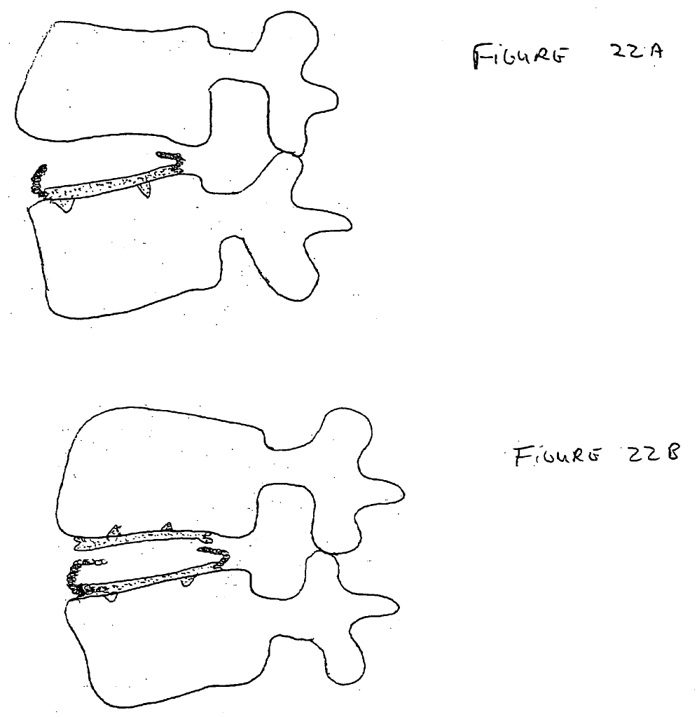

FIG. 20A is a coronal cross section through yet a different alternative embodiment of the invention, wherein the spacer component articulates with both ADR EPs. An eclipse upper surface of the upper portion of the spacer allows rotation of the upper ADR EP relative to the spacer. The eclipse shape does not permit spinal flexion, extension, or lateral bending. The round shape of the lower portion of the spacer component permits spinal rotation, flexion, extension, lateral bending, and rotation.

FIG. 20B is a coronal cross section of an alternative embodiment of the ADR drawn in FIG. 20A. Round surfaces on the superior and inferior portions of the spacer component permit spinal rotation, lateral bending, flexion, and extension through articulations at both ADR EPs and the spacer. FIG. 20C is a coronal cross section of an alternative embodiment of the ADR drawn in FIG. 20A. The shape of the superior portion of the spacer does not permit spinal motion in any direction through the articulation between the ADR EP and the spacer.

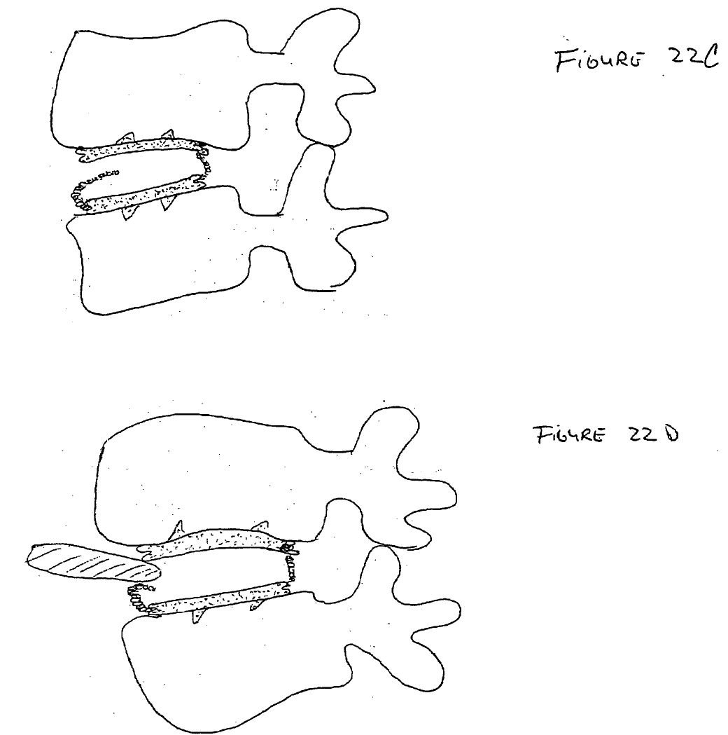

FIG. 21A is a sagittal cross section of the spine and another embodiment of the ADR. The ADR has two ADR endplates (EP) and an elastic component that is attached lo the posterior and lateral sides of the ADR EPs. The elastic component is not attached to the anterior portion of top ADR EP. The elastic component could be a mesh containing horizontal Nitinol hoops and other vertical Nitinol members such as wires. The opening between the anterior portion of the elastic component and the anterior portion of one of the ADR EPs allows room for insertion of a tool to press fit the ADR EPs into the vertebrae.

FIG. 21B is a drawing of the next step in the sequence of insertion of the ADR drawn in FIG. 21A. A partially dehydrated hydrogel is inserted through the opening between the elastic component and the ADR EP. FIG. 21C is a sagittal cross section of the spine and the completed ADR drawn in FIG. 21A. The anterior portion of the elastic component is connected to the anterior portion of both ADR EPs. The hydrogel imbibes body fluid and expands after insertion between the ADR EPs. The hydrogel component is drawn wedge shaped to fit the natural disc space anatomy. Other elastomeric or cushioning materials may alternatively be used in all applicable embodiments. The posterior portion of the elastic component is flexed more than the anterior portion of the elastic component. The flexed posterior portion of the elastic component aids increased spinal flexion relative to spinal extension.

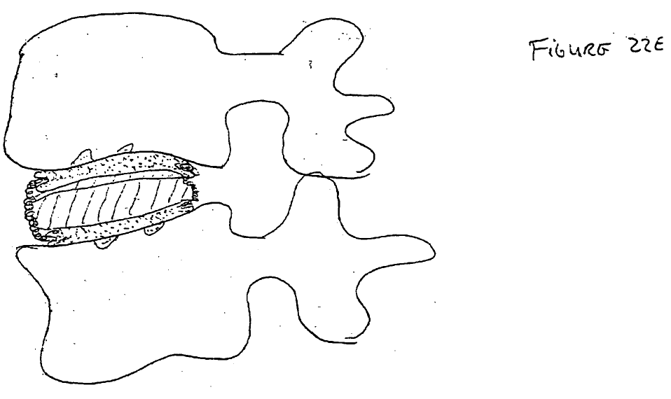

FIG. 22A is a sagittal cross section of the spine and another embodiment of the ADR. A first ADR EP, with an attached elastic component is press fit into a first vertebra. FIG. 22B is a sagittal cross section of the spine and the next step in the insertion of the ADR drawn in FIG. 22A. The second ADR EP is press fit to the second vertebra. FIG. 22C is a sagittal cross section of the spine and the next step in the insertion of the ADR drawn in FIG. 22B. The posterior portion of the elastic component is attached to the posterior portion of the second ADR EP. Projections from the second ADR EP could fit through holes in the elastic component to help align the elastic component and the ADR EP. FIG. 22D is a sagittal cross section of the spine and the next step in the insertion of the ADR drawn in FIG. 22C. A partially dehydrated hydrogel is inserted into the space between the ADR EPs. FIG. 22E is a sagittal cross section of the spine and the completed ADR drawn in FIG. 22D. The elastic component is attached to all sides of both ADR EPs. The hydrogel imbibes body fluids and expands.

I claim:

1. Artificial disc replacement (ADR) apparatus, comprising:
an endplate that articulates with a cooperating component; and
wherein the endplate, or the endplate and the cooperating component, are physically configured for assembly within an intervertebral disc space.

2. The ADR apparatus of claim 1, wherein:
the endplate, or the endplate and the cooperating component, are composed of dissimilar materials.

3. The ADR apparatus of claim 1, wherein:
the endplate is composed of Nitinol or other shape-memory material.

4. The ADR apparatus of claim 3, wherein the Nitinol or other shape-memory material is used to form projections that diverge or converge after insertion in the disc space.

5. The ADR apparatus of claim 1, wherein:
the endplate includes an articulating component composed of chrome cobalt or another metal alloy.

6. The ADR apparatus of claim 1, wherein the endplate is provided as two separate components that are physically configured for assembly within an intervertebral disc space.

7. The ADR apparatus of claim 5, wherein each of the separate components are press-fit into a vertebral body.

8. The ADR apparatus of claim 5, wherein the separate components are connected through a snap-fit engagement.

9. The ADR apparatus of claim 5, wherein the separate components are connected through a hinge.

10. The ADR apparatus of claim 1, wherein:
the endplate includes an articulating component that is treaded into the endplate.

11. The ADR apparatus of claim 1, wherein:
the endplate includes an articulating component that is press-fit into the endplate.

12. The ADR apparatus of claim 1, wherein:
the endplate includes an articulating component that is press-fit through a Morse-taper type joint.

13. The ADR apparatus of claim 1, wherein the cooperating component is a spacer.

14. The ADR apparatus of claim 13, wherein the spacer is rotated or otherwise manipulated to achieve a vertebral distraction function.

15. The ADR apparatus of claim 13, wherein the spacer is contained within a disc space using a clip or other retaining element.

16. The ADR apparatus of claim 13, wherein the spacer is contained within a disc space using a clip or other retaining element.

17. The ADR apparatus of claim 13, wherein the spacer is contained within a disc space using a mesh or elastic component.

18. A method of implanting an artificial disc replacement (ADR) into an intervertebral disc space, comprising the steps of:
providing an endplate constructed from first and second components;
installing the first component into an intervertebral disc space; and
installing the second component into the disc space by attaching the second component to the first component, thereby assembling the endplate in situ.

19. The method of claim 18, wherein the first and second components are comprised of dissimilar materials.

20. The method of claim 18, further including a spacer component which is also assembled in situ.

21. A method of implanting an artificial disc replacement (ADR) into an intervertebral disc space, comprising the steps of:
providing an endplate constructed from first and second components;
installing the first component into an intervertebral disc space; and
installing the second component into the disc space by attaching the second component to the first component, thereby assembling the endplate in situ.
